(19) United States
(12) Patent Application Publication
(10) Pub. No.: US 2025/0123253 A1
(43) Pub. Date: Apr. 17, 2025

(54) BORATE DETECTOR COMPOSITION AND ASSAY SOLUTION

(71) Applicant: WATER LENS, LLC, Houston, TX (US)

(72) Inventors: Justin M. DRAGNA, Austin, TX (US); Tyler WEST, Austin, TX (US)

(21) Appl. No.: 18/779,776

(22) Filed: Jul. 22, 2024

Related U.S. Application Data
(63) Continuation of application No. 17/704,702, filed on Mar. 25, 2022, now abandoned.
Application No. 17/704,702 is a continuation of application No. 16/792,525, filed on Feb. 17, 2020, now abandoned.
Application No. 16/792,525 is a continuation of application No. 15/436,927, filed on Feb. 20, 2017, now abandoned.
Application No. 15/436,927 is a continuation of application No. 14/446,800, filed on Jul. 30, 2014, now abandoned.
(60) Provisional application No. 61/970,194, filed on Mar. 25, 2014.
(60) Provisional application No. 61/860,220, filed on Jul. 30, 2013.

(51) Int. Cl. G01N 31/22; G01N 33/18

(52) U.S. Cl. CPC G01N 31/221; G01N 31/22; G01N 33/182; Y10T 436/15

(57) ABSTRACT

A composition and an assay solution for the determination of dissolved borate concentration comprising a catechol dye, a solubilizing agent, and a buffer are described. The composition and assay solution may further comprise a solubilizing agent. The catechol dye acts as a chemical borate sensor. The chemical borate sensor changes its optical properties upon binding to borate. The multivalent cation chelator binds multivalent cations present in a sample being analyzed. The buffer prevents changes in pH. The solubilizing agent aids in solubilizing the catechol dye, multivalent cation chelator, and/or the buffer.

### CROSS-REFERENCE TO RELATED APPLICATIONS

This application is a continuation of U.S. application Ser. No. 17/704,702 filed Mar. 25, 2022, which is a continuation of U.S. application Ser. No. 16/792,525 filed Feb. 17, 2020 (now abandoned), which is a continuation of U.S. application Ser. No. 15/436,927 filed Feb. 20, 2017 (now abandoned), which is a continuation of U.S. application Ser. No. 14/446,800 filed Jul. 30, 2014 (now abandoned), which claims priority to U.S. Prov. Application 61/860,220 filed Jul. 30, 2013 and U.S. Prov. Application 61/970,194 filed Mar. 25, 2014, each of which are herein incorporated by reference in their entirety without disclaimer.

### FIELD OF THE INVENTION

This invention relates to environmental chemistry and quantitative chemical analysis.

### DESCRIPTION OF RELATED ART

Campana et al. Analyst July 1992, Vol. 117 describes a spectrofluoremetric method for the determination of boron in soils, plants and natural waters with Alizarin Red S. The method employs a spectfluorometer for the fluorometric detection. The method measures the fluorescence excitation and emission spectra of the Boron-Alizarin RedS complex to determine boron concentration.

Campana et al. Analyst August 1994, Vol. 119 describes a method for the spectrofluoremetric determination of molybdenum with Alizarin Red. S in the presence of hexadecyltrimethylammonium bromide. The method measures the fluorescence excitation and emission spectra of the MO-ARS complex.

Arimori et al., Chemical Communications 2001, 2018-2019 describes fluorescent sensors for boronic and boric acids. The sensors comprise anthracenic tertiary amines as sensor molecules.

Villamil-Ramos and Yatsimirsky Chemical Communications 2011, 2694-2696 describe a method for the fluorometric detection of pyrophosphate by interaction with alizarin red S-dimethyltin(IV) complex. The detection method measures pyrophosphate dimethyltin(IV)-ARS complexes by the fluorescence at 610 nm

Tomsho and Benkovic, The Journal of Organic Chemistry 2012, Vol. 77, 2098-2106 describes the mechanism of the reaction between phenylboronic acid and Alizarin Red S. Boronic acid, or a boronate anion form a boronic ester with a 1,2-diol, whose fluorescence may be measured.

### SUMMARY

A composition and an assay solution for the determination of dissolved borate concentration comprising a catechol dye, a multivalent cation chelator, and a buffer are described. In some embodiments, the composition further comprises a solubilizing agent. The catechol dye acts as a chemical borate sensor. The chemical borate sensor changes its optical properties upon binding to borate. The multivalent cation chelator binds multivalent cations present in a sample being analyzed. The buffer prevents changes in pH. In some embodiments, the buffer displays a solubility in water greater than 200 g/L. In particular embodiments, the solubilizing agent increases the solubility of the dye, multivalent cation chelator, and/or the buffer. In one embodiment, the operable pH range for borate concentration determination is from about 6 to about 8. In other embodiments, the operable pH range for borate concentration determination is from about 4 to about 12. The borate concentration is measureable in waters with high total dissolved solids.

In some embodiments, the catechol dye is Alizarin Red S. In some embodiments, the multivalent cation chelator is EDTA. In some embodiments, the buffer is imidazole. In preferred embodiments, EDTA binds metals present in a sample being analyzed. In other embodiments, EDTA minimizes errors in measured borate concentration. In yet other embodiments, Alizarin Red S reacts with borate to form complex 1-BO4. In some embodiments, the borate concentration is measureable in waters with high total dissolved solids.

In some aspects of the invention, the solubilizing agent is a cyclodextrin. In particular embodiments, the cyclodextrin is an α-cyclodextrin. In other embodiments, the cyclodextrin is a β-cyclodextrin. In other embodiments, the cyclodextri is a γ-cyclodextrin. In further embodiments, the cyclodextrin is an alkylated cyclodextrin. In particular embodiments, the cyclodextrin is hydroxypropyl β-cyclodextrin. In other embodiments, the solubilizing agent may be a surfactant, a crown ether, a polyethylene glycol, or other excipient. In some embodiments, the solubilizing agent is present in a range of from 1% to 10%.

The assay solution may include solution dispensed into multi-well plates. The assay solution includes freeze-dried solution. A kit for the determination of dissolved borate concentration comprising a catechol dye, a multivalent cation chelator, a solubilizing agent, and a buffer in a container containing multiple test locations, preferably a 96-well plate is described. The kit comprises the assay solution described. A method of determining the borate concentration in water, comprising contacting the sample with any one of the compositions of claims 1-27 or any one of the assay solutions of claims 28 to 57, and determine the concentration of borate in the sample is described. In some embodiments, the method comprises employing the inventive assay solution and a fluorometric detector. In some embodiments, the sample for determination of borate concentration is an aqueous sample. Other non-limiting types of samples for which borate concentration may be determined include soils and other solids, gels, slurries, suspensions, tissues and the like.

One skilled in the art recognizes that the concentrations of different compounds will depend on the detector. One skilled in the art recognizes that the concentrations required to obtain a linear response will vary.

The concentrations can be adjusted and the detector path length can be adjusted. For example a decrease in the path length will allow for an increase in the concentration. Increasing the path length will allow for a decrease in the concentration.

Details associated with the embodiments described above and others are presented.

### BRIEF DESCRIPTION OF THE DRAWINGS

The following drawings illustrate by way of example and not limitation. For the sake of brevity and clarity, every feature of a given structure may not be labeled in every figure in which that structure appears. Identical reference numbers do not necessarily indicate an identical structure. Rather, the same reference number may be used to indicate a similar feature or a feature with similar functionality, as may non-identical reference numbers.

Unless otherwise noted, the figures are drawn to scale, meaning that the sizes of the depicted items are accurate relative to each other for at least the embodiments depicted in the figures.

FIG. 1A is a drawing of the chemical reaction that occurs upon binding of Alizarin Red S to borate.

FIG. 1B is a drawing of the sequestration of multivalent ions by coordination to EDTA.

FIG. 1C is a drawing of the imidazole buffering mechanism.

FIG. 2 is a calibration curve that plots change in absorbance at 520 nm as a function of borate concentration.

### DETAILED DESCRIPTION

Various features and advantageous details are explained more fully with reference to the non-limiting embodiments that are illustrated in the accompanying drawings and detailed in the following description. It should be understood, however, that the detailed description and the specific examples, while indicating embodiments of the invention, are given by way of illustration only, and not by way of limitation. Various substitutions, modifications, additions, and/or rearrangements will become apparent to those of ordinary skill in the art from this disclosure.

The terms “a” and “an” are defined as one or more unless this disclosure explicitly requires otherwise.

The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a composition and/or an assay solution that “comprises,” “has,” “includes” or “contains” one or more elements possesses those one or more elements, but is not limited to possessing only those one or more elements. Likewise, an element of a system or composition that “comprises,” “has,” “includes” or “contains” one or more features possesses those one or more features, but is not limited to possessing only those one or more features.

Furthermore, a structure or composition that is configured in a certain way is configured in at least that way, but may also be configured in ways that are not listed. Metric units may be derived from the English units provided by applying a conversion and rounding to the nearest millimeter.

The feature or features of one embodiment may be applied to other embodiments, even though not described or illustrated, unless expressly prohibited by this disclosure or the nature of the embodiments.

Any embodiment of any of the disclosed container assemblies and compositions can consist of or consist essentially of—rather than comprise/include/contain/have—any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb.

As used herein, high total dissolved solids includes values above 60,000 mg/L. In the following description, numerous specific details are provided to provide a thorough understanding of the disclosed embodiments. One of ordinary skill in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The assay can be used as a field test for the determination of dissolved borate in aqueous solutions. In one embodiment, the assay is designed to test produced water at oil and gas sites. In particular instances, corrosive chemicals such as sulphuric acid (used in other commercially available assays) are avoided. The assay can be performed in any aqueous solution. In one embodiment of the present invention, the assay was performed in waters with extremely high total dissolved solids (TDS), where a large percentage of the TDS are multivalent metals such as, but not limited to, Ca⁺², Mg⁺², Fe⁺², and Fe⁺³.

The assay comprises a solution as made up of a catechol dye, a multivalent cation chelator and a buffer. Any catechol dye known to those of skill in the art may be used. Examples of such catechol dyes comprise Alizarin Red S and pyrocatechol violet. The multivalent cation chelator may comprise EDTA, 1,2-bis(o-aminophenoxy)ethane-N,N,N′,N′-tetraacetic acid (BAPTA), Quin-2, BAPTA-AM, Fura-1, Fura-2, Fura-3, 1,2-Bis(2-Aminophenoxy)ethane-N,N,N′,N′-tetraacetic acid, APTRA, 5F-APTRA, 2-Hydroxyisoquinoline-1,3(2H,4H)-dione, other different salts thereof, or any multivalent cation chelator known to those of skill in the art. Buffers may comprise imidazole, phosphate, HPEES, citrate, or other buffers known to those of skill in the art.

In some embodiments, the catechol dye exhibits a maximal borate sensitivity at a given pH. In some embodiments, a chelator is employed such that the chelator pKa is below the catechol dye borate maximal sensitivity pH. In a particular embodiment, the dye is Alizarin Red S and exhibits a maximal sensitivity at a pH of about 7.2. In a further embodiment, a metal chelator that blocks metal ions from binding to/and or interfering with Alizarin Red S has a pKa below 7.2. In this particular embodiment, the chelator is BAPTA (CAS number 85233-19-8). However, other chelators such as Fura-2 (CAS 112694-64-7) may also be used.

In preferred embodiments, a chelator is employed that is fully deprotonated at the pH necessary for the dye to be sensitive. If a chelator is protonated at a pH of interest, as in the case of EDTA at pH 7.2, the addition of metals may result in the chelator releasing protons, thereby causing the pH to change and/or necessitate the use of very high buffer concentrations. A change in pH may cause the assay to lose accuracy because the dye may change color in response to pH in a similar manner to how it changes color in response to borate. To stop this change, buffer can be added. However, the amount of buffer that can be added is limited by the solubility of the buffer. Although the solubility of buffers varies, it is preferred to use a buffer concentration of less than or equal to approximately 1M.

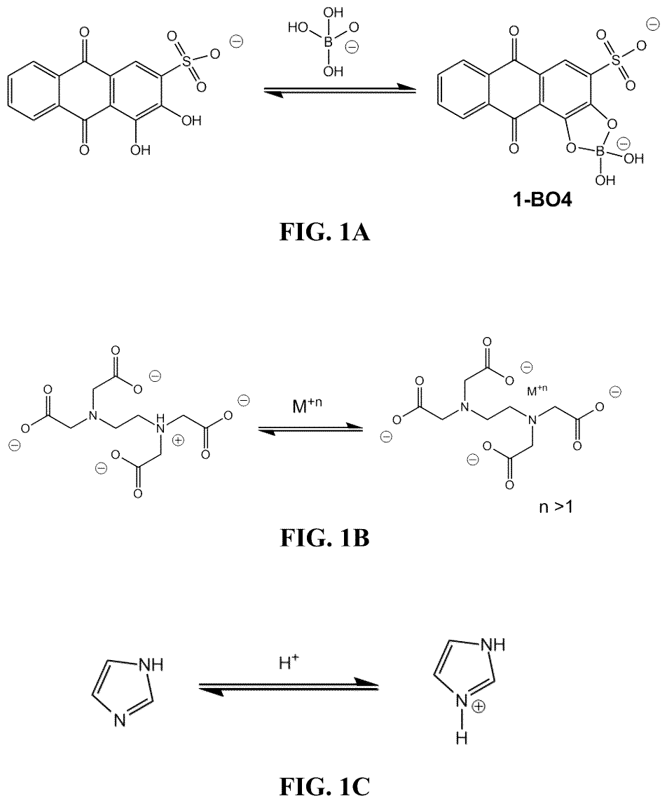

In a preferred embodiment, the assay comprises a solution as made up of compound 1 (Alizarin Red S), compound 2 (EDTA), compound 3 (imidazole) and hydroxypropyl β-cyclodextrin in water at a pH between 6 and 8 (FIGS. 1A-1C). Alizarin Red S is the chemical sensor and it changes its optical properties when it reacts with borate to form complex 1-BO4, which allows for the development of calibration curves for use in the determination of the concentration of borate in samples with unknown concentration. EDTA is a masking agent and binds any metals present in the high TDS sample being analyzed. The EDTA is important because metals can also bind to Alizarin Red S and change its optical properties, which can result in errors in the determination of the concentration of boron in unknown samples. Imidazole is the buffer. The buffer is important because Alizarin Red S changes its optical properties in response to pH. Hydroxypropyl β-cyclodextrin increases the solubility of the dye. One skilled in the art recognizes that Alizarin S can work in a variety of pH ranges, including from 1-12. In a particular embodiment, a range of 6-8 has been found to be useful. Thus, without a buffer, the pH would change on the addition of a sample, resulting in an increase in error for the assay.

In one embodiment, the assay is dispensed into 96-well plates and freeze-dried. The freeze-drying allows the assay to rapidly dissolve on the addition of a sample for analysis. Freeze dried samples are hygroscopic; thus, the 96-well plates with the freeze-dried assay are stored in mylar bags filled with nitrogen and containing a dessicant. Hydroxypropyl β-cyclodextrin prevents the dye from precipitating out of solution when temperature decreases.

The assay can be conducted as a single assay in an appropriate container. It is advantageous to be able to have a container that allows multiple testing at the same time. One skilled in the art recognizes that multiple well containers are well known in the art and can be used for multiple tests. In one embodiment, a 96-well plate is used. The assay solution includes solution dispensed into 96-well plates. The assay solution includes freeze-dried solution. A kit for the determination of dissolved borate concentration comprising a catechol dye, a multivalent cation chelator, and a buffer in a container containing multiple test locations, preferably a 96-well plate is described. The kit comprises the assay solution described. A method of determining the borate concentration in water, comprising employing the assay solution is described.

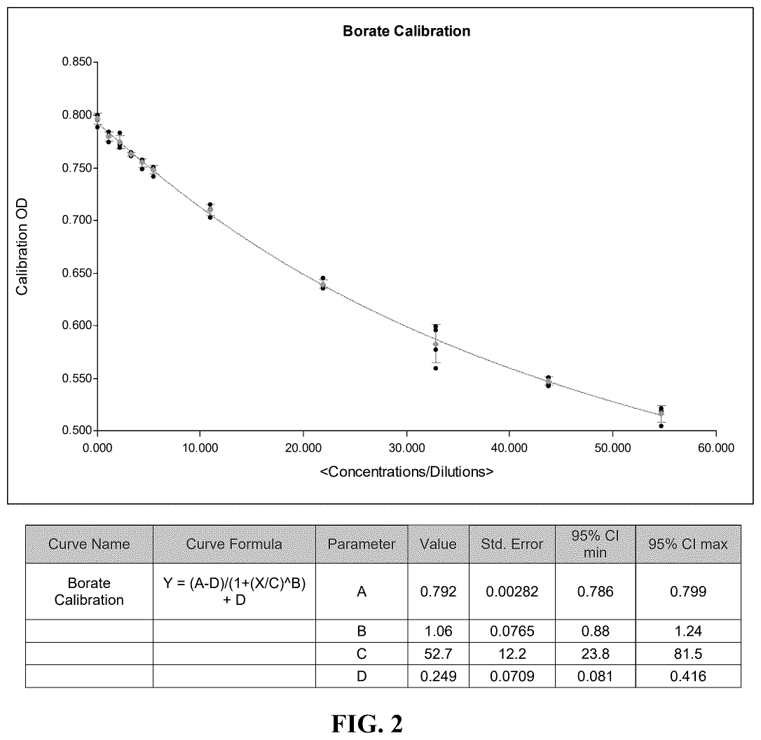

One skilled in the art recognizes that the absorbance can be read in between 200 and 620 nm. In one particular embodiment, it is found that 520 nm works well and thus, calibration curves were developed between 0 and 60 mg/L of borate by plotting the change in the absorbance at 520 nm as a function of the change in borate concentration. The resulting calibration data was fit with a curve using nonlinear regression in Gen5 software (FIG. 2). The calibration curve is used for determining the concentration of borate in samples of unknown concentration.

Any optical reader can be used to detect the results of the assay. Most optical readers will have their own software package to help normalize the curves. In one aspect of the present invention, the optical data for the assay is collected using a commercially available plate reader from Biotek. The Biotek plate reader comes with a software package called Gen5. The Gen5 software is programmed so that a user can click a button to start an experiment. Once the experimented is started, the program automatically reads the wavelength of the assay at 520 nm and plots the absorbance value on the calibration curve to determine the concentration of the unknown sample.

### EXAMPLES

The sensor solution was prepared by combining 185.5 g disodium ethylenediaminetetraacetate (EDTA) dihydrate with 69.326 g imidazole free base in 700 mL distilled water. The solution was heated until all constituents went into solution, and the pH verified to be 7.19. From a concentrated solution of Alizarin Red S (58.12 mM in distilled water), 10.32 mL were added. A large portion of the dye appeared to precipitate, but returned to solution with gentle heating. A second batch of the sensor buffer solution was prepared as above, using 185.468 g disodium EDTA dihydrate and 70.582 g imidazole free base. Both solutions were then transferred to a 2000 mL volumetric flask and diluted to the mark with distilled water, resulting in the final sensor solution: 0.6 mM ARS, 1.0 M imidazole, 0.5 M EDTA, pH 7.2.

TABLE 1

| Well ID | Name | Well | Conc/Dil | A520 | Count | Mean | Std Dev | CV (%) |
|---|---|---|---|---|---|---|---|---|
| STD1 | Boron | A1 | 0 | 0.789 | 4 | 0.796 | 0.005 | 0.652 |
| | Standards | | | | | | | |
| | | B1 | 0 | 0.8 | | | | |
| | | C1 | 0 | 0.8 | | | | |
| | | D1 | 0 | 0.796 | | | | |
| STD2 | Boron | A2 | 1.1 | 0.774 | 4 | 0.78 | 0.004 | 0.538 |
| | Standards | | | | | | | |
| | | B2 | 1.1 | 0.78 | | | | |
| | | C2 | 1.1 | 0.784 | | | | |
| | | D2 | 1.1 | 0.781 | | | | |
| STD3 | Boron | A3 | 2.19 | 0.769 | 4 | 0.774 | 0.006 | 0.785 |
| | Standards | | | | | | | |
| | | B3 | 2.19 | 0.783 | | | | |
| | | C3 | 2.19 | 0.773 | | | | |
| | | D3 | 2.19 | 0.772 | | | | |
| STD4 | Boron | A4 | 3.29 | 0.761 | 4 | 0.763 | 0.002 | 0.224 |
| | Standards | | | | | | | |
| | | B4 | 3.29 | 0.763 | | | | |
| | | C4 | 3.29 | 0.765 | | | | |
| | | D4 | 3.29 | 0.764 | | | | |
| STD5 | Boron | A5 | 4.38 | 0.749 | 4 | 0.755 | 0.004 | 0.523 |
| | Standards | | | | | | | |
| | | B5 | 4.38 | 0.756 | | | | |
| | | C5 | 4.38 | 0.758 | | | | |
| | | D5 | 4.38 | 0.756 | | | | |
| STD6 | Boron | A6 | 5.48 | 0.742 | 4 | 0.748 | 0.004 | 0.56 |
| | Standards | | | | | | | |
| | | B6 | 5.48 | 0.75 | | | | |
| | | C6 | 5.48 | 0.751 | | | | |
| | | D6 | 5.48 | 0.75 | | | | |
| STD7 | Boron | A7 | 10.95 | 0.703 | 4 | 0.71 | 0.005 | 0.709 |
| | Standards | | | | | | | |
| | | B7 | 10.95 | 0.711 | | | | |
| | | C7 | 10.95 | 0.715 | | | | |
| | | D7 | 10.95 | 0.711 | | | | |
| STD8 | Boron | A8 | 21.9 | 0.636 | 4 | 0.639 | 0.004 | 0.606 |
| | Standards | | | | | | | |
| | | B8 | 21.9 | 0.645 | | | | |
| | | C8 | 21.9 | 0.638 | | | | |
| | | D8 | 21.9 | 0.639 | | | | |
| STD9 | Boron | A9 | 32.85 | 0.56 | 4 | 0.583 | 0.018 | 3.116 |
| | Standards | | | | | | | |
| | | B9 | 32.85 | 0.596 | | | | |
| | | C9 | 32.85 | 0.599 | | | | |
| | | D9 | 32.85 | 0.577 | | | | |
| STD10 | Boron | A10 | 43.8 | 0.543 | 4 | 0.548 | 0.004 | 0.753 |
| | Standards | | | | | | | |
| | | B10 | 43.8 | 0.545 | | | | |
| | | C10 | 43.8 | 0.551 | | | | |
| | | D10 | 43.8 | 0.551 | | | | |
| STD11 | Boron | A11 | 54.75 | 0.505 | 4 | 0.516 | 0.008 | 1.478 |
| | Standards | | | | | | | |
| | | B11 | 54.75 | 0.519 | | | | |
| | | C11 | 54.75 | 0.522 | | | | |
| | | D11 | 54.75 | 0.519 | | | | |

Table Concentrations are in mg/L.

1-95. (canceled)

96. A method for determining dissolved borate concentration in an aqueous sample, the method comprising:
contacting an aqueous sample obtained at an oil and gas site with a freeze dried borate assay composition comprising a catechol dye, a multivalent cation chelator, a buffer, and a solubilizing agent, and
determining the concentration of dissolved borate in the aqueous sample,
wherein the freeze dried borate assay composition is comprised in a kit comprising a multi-well plate comprising at least a first, second, third, and fourth well,
wherein the freeze dried borate assay composition is included in the first, second, third, and fourth wells in increasing amounts such that the fourth well includes more freeze dried borate assay composition than the third well, the third well includes more freeze dried borate assay composition than the second well, and the second well includes more freeze dried borate assay composition than the first well, and
wherein the aqueous sample is contacted with the freeze dried borate assay composition present in each of the first, second, third and fourth wells.

97. The method of claim 96, wherein the catechol dye is a chemical borate sensor.

98. The composition of claim 97, wherein the chemical borate sensor changes its optical properties upon binding to borate.

99. The method of claim 98, wherein the catechol dye comprises pyrocatechol violet.

100. The method of claim 96, further comprising obtaining the kit and providing the kit to the oil and gas site.

101. The method of claim 100, wherein the kit comprises an optical reader.

102. The method of claim 101, wherein determining the amount of borate comprises positioning the multi-well plate in the optical reader at the oil and gas site, collecting optical data and plotting absorbance values on a borate calibration curve.

103. The method of claim 102, wherein optical reader comprises a fluorometric detector.

104. The method of claim 96, wherein contacting the aqueous sample with the freeze dried borate assay composition dissolves the composition, forming a solution comprising the aqueous sample, the catechol dye, the multivalent cation chelator, the buffer, and the solubilizing agent.

105. The method of claim 104, wherein the pH of the solution is about 4 to about 12.

106. The method of claim 105, wherein the pH of the solution is about 6 to about 8.

107. The method of claim 96, wherein the aqueous sample has dissolved solids.

108. The method of claim 107, wherein the dissolved solids comprise Ca⁺², Mg⁺², Fe⁺², and Fe⁺³.

109. The method of claim 96, wherein the solubilizing agent is present in a range of from 1% to 10%.

110. The method of claim 96, wherein the multivalent cation chelator has a pKa value below the catechol dye maximal sensitivity pH.

111. The method of claim 96, wherein the buffer solubility is greater than 200 g/L.

112. The method of claim 96, wherein the solubilizing agent increases the solubility of the dye, multivalent cation chelator, the buffer, or a combination thereof.

113. The method of claim 96, wherein the kit is obtained by:
dispensing an unfrozen borate assay composition comprising the catechol dye, the multivalent cation chelator, the buffer, and the solubilizing agent into the wells of the multi-well plate;
subjecting the multi-well plate comprising the borate assay composition to conditions sufficient to freeze and dry the unfrozen borate assay composition; and
storing the multi-well plate in a bag.

114. The method of claim 96, wherein the solubilizing agent comprises a polyethylene glycol.

115. The method of claim 96, wherein the solubilizing agent comprises a surfactant.
